(19) United States
(12) Patent Application Publication
(10) Pub. No.: US 2006/0147896 A1
(43) Pub. Date: Jul. 6, 2006

(54) Method and device for manipulating samples

(75) Inventors: Guntram Schnetz, Biedermannsdorf (AT); Heinz Redl, Vienna (AT); Kurt Zatloukal, Graz (AT)

Correspondence Address: FULBRIGHT & JAWORSKI L.L.P., 600 CONGRESS AVE., SUITE 2400, AUSTIN, TX, 78701, US

(73) Assignee: ORIDIS BIOMED Forschungs-und Entwicklings GmbH, Graz (AT)

(21) Appl. No.: 10/533,493

(22) PCT Filed: Oct. 30, 2003
(86) PCT No.: PCT/AT03/00324

§ 371 Date: Apr. 29, 2005

(30) Foreign Application Priority Data
Oct. 31, 2002 (AT) A 1649/2002

(51) Int. Cl. C12Q 1/00; G06F 19/00

(52) U.S. Cl. 435/4; 702/19

(57) ABSTRACT

The invention relates to a method and a device for manipulating samples, in particular tissue samples, with at least one needle (3) for punching samples from preparations (5), in particular prepared tissue specimen, at defined positions and a control device (13) to control the needle (3). The aim of the invention is to provide a method and a device, which allow a simple selection of desired punch positions on the preparation (5) and enable a rapid, simple and automatic manipulation of samples. To achieve this, the device is equipped with a camera (14) to take images of the surface of the tissue specimen (5), furthermore a device for superimposing the images captured of the preparations (5) with digital, microscopic images of sections of these preparations (5) that have been archived in a storage device (11), furthermore a monitor (12) to display the superimposed images and furthermore a device provided to place markings for the determination of the defined sample-punching positions, which is connected with the control device (13).

The invention relates to a method for manipulating samples, in particular tissue samples, wherein at defined positions, samples are punched-out of preparations, in particular prepared tissue specimens, by means of needles and said samples are inserted into punched-out holes in sample carriers.

In addition, the invention relates to a device for manipulating samples, in particular tissue samples, comprising at least one needle for punching samples out of preparations, in particular prepared tissue specimens, at defined positions and a control device for controlling the needle.

The term “preparations” thereby particularly includes tissue specimens from humans or animals. However, other materials and substances, such as embedded cell- or bacteria suspensions, but also plants and sections of plants respectively, can be analysed and their samples manipulated.

Biological tissues are removed frequently from human or animal organs for medical as well as scientific purposes and, following a series of preparation- and processing steps, used in various assays, for instance to identify diseases, changes in tissue or for the assessment of the progression of therapies. Thereby, the removed tissue is usually embedded in paraffin, synthetic material or a different, comparable material and one or more specific samples punched-out of said embedded tissue specimens. For this purpose cylindrical tissue samples are punched-out using needles. Said punched-out tissue samples are then inserted into equivalent sized punched-out holes in a sample carrier, also prepared with the help of needles. The sample carrier normally consists of paraffin, synthetic material or a similar material. Furthermore, thermoplastic materials, which are gel-like at room temperature and solidify at lower temperatures, for instance −10° C., are well established for the embedding of preparations, in particular tissue specimens, but also for the insertion of samples. Frozen samples in particular can be prepared with such materials. Needles with an outer diameter essentially the same as the inner diameter of the needles used to punch-out tissue samples from the tissue specimens are used to create the holes in the sample carrier. The punched-out tissue sample thus fits exactly into the punched-out hole in the sample carrier. In this way, so-called tissue arrays or micro arrays containing a large number of adjacently arranged tissue samples are prepared. In this way, sections are prepared from such tissue sample arrays, normally using a microtome, and used for histological or pathological analyses. Several hundred tissue samples can be arranged at the same time in sample carriers possessing an area of for instance 3×4 cm. The number of individual samples resulting from the preparation of sections and requiring evaluation is correspondingly high. Because of the enormous number of tissue samples the manipulation of the tissue samples should be carried out as rapidly as possible and be automated. For that purpose, devices for the manipulation of tissue samples have been developed with the help of which such tissue arrays can be produced as quickly as possible and with as high a degree of accuracy as possible.

U.S. Pat. No. 6,103,518 A for example describes a device of the present kind, for the manipulation of samples, where holes are punched-out from sample carriers by means of a needle and tissue samples are punched-out from prepared tissue specimen with a further needle and said tissue samples inserted into the punched-out holes in the sample carrier. Those positions on the tissue specimen from which tissue samples should be punched-out are normally selected manually, thereby significantly slowing-down the manipulation process. Furthermore, the selection of those positions on the tissue specimen from which tissue samples will be punched-out is mostly carried out under optical control by a person skilled in the medical art. For this, tissue sections of the tissue sample can be used to assist in the selection of the punching positions.

DE 198 15 400 A1 relates to a device and a method for sample incorporation to polymeric carrier materials with which, through using multiple separation tools, for instance puncture capillaries that are individually controllable, samples can be sequentially punched-out and then simultaneously deposited on a target substrate. The device enables the simultaneous processing of a large number of samples. Further described is the combination of the sample incorporation device with an imaging system which makes possible the automation and acceleration of the sample incorporation process. The selection of chosen punch positions can be facilitated by the image of the surface of the preparation or equivalent, from which the samples will be punched-out.

In order to facilitate the selection of the chosen punch positions on the preparation, sections are frequently prepared from the preparation and macroscopic images made that are employed by the relevant person skilled in the art to select the punch positions. Thereby, such macroscopic images provide limited additional information on the preparation itself.

The aim of the present invention is to create a method for the manipulation of samples, in particular tissue samples, of the kind described above, which enables as fast a selection of the desired punch positions as possible and which is as simple and accomplished as quickly as possible. Furthermore, according to the present invention, samples should be prepared that provide as high a level of quality and specificity as possible.

A further task of the present invention is to create a device for the manipulation of samples, in particular tissue samples of the kind described, which permits as simple as possible a selection and definition of the desired punch positions on the preparation and which permits automatic or semi-automatic manipulation. Furthermore, the device should be as simple and economically to build as possible and require as little maintenance as possible. The device should furthermore be capable of manipulating a large number of samples. The disadvantages of the state of the art should be avoided or at least reduced.

The first task of the invention is solved by superimposing at least one digital microscopic image of a section of a preparation on an image of the surface of this preparation and by the placing of markings on the superimposed image which define the desired positions from which samples will be automatically punched-out and inserted into holes in the sample carrier. The superimposing of at least one digital microscopic section image from the preparation simplifies the selection of the optimum punch positions on the preparation and thus increases the quality of the resulting samples and as a result the quality of diagnoses as well as test results. Through the placing of markings at the chosen punch positions the samples need not be immediately punched-out after the selection of a particular position, rather, after the placing of all markings, they can be punched-out in an automated process. The availability of microscopic section images of the corresponding preparations provides detailed information for the corresponding person trained in the medical arts who selects the desired punch positions.

Because the field of view of a microscope is too small at the appropriate magnification in order to display the entire section of the preparation, the digital microscopic images can also be separated into segments which are joined together before superimposing the surface image of the preparation.

To further improve the quality of the resulting samples through optimal information about the preparation it is planned that the digital microscopic images or their segmented-images are straightened or freed from artefacts.

To enable a punching process after placing of all markings, the markings or their coordinates are stored in a database together with an identifier for the preparation. In this way, a large number of preparations can accordingly be processed and markings placed on the preparations and subsequently the corresponding samples punched-out of the preparations in an efficient automated process, inserted into the corresponding sample carrier which will thereafter be further applied to the corresponding tests. The identifier for the preparation can for example be displayed as a barcode or equivalent on the preparation or on the paraffin block containing the preparation respectively.

In order to further facilitate the choice of punch positions multiple digital section images can be selected. These section images, taken before the manipulation process, are likewise stored in the appropriate database together with the identifier for the preparation.

In order to facilitate the placing of markings at the desired punch positions the display scale of the section images, surface images and/or superimposed images respectively, can be adjusted. Consequently, the person skilled in the medical art can enlarge the area of interest or can obtain a complete overview by appropriate reduction of the magnification.

A simplification of the present method is also achieved by enabling the section images, surface images and/or superimposed images respectively, to be displaced and their colours modified. By changing the colours, specific effects are obtained that facilitate the interpretation of section images as well as of surface images or superimposed images.

According to a further feature of the method, the degree of translucency of the superimposed surface image in terms of the section image can be adjusted in order to optimise the display of the superimposed image. This so-called Alpha Value determines how strongly the surface image of the preparation should be visible through the microscopic section image.

Further, the transparency of the section image can be altered to enable the display of the superimposed image to be optimised.

In order to provide the person skilled in the medical art with access to information about a given preparation an identifier for the corresponding patient information or equivalent can be displayed together with the superimposed image. This patient information or equivalent is advantageously stored in a database and is displayed for instance to support the process of choosing the punch positions.

The positioned markings can be for instance labelled through a consecutive numbering. In this way all markings are unambiguously identifiable.

In order to enable the selection of punch positions to also be reversed, placed markings can be selected and erased.

In order to enable already selected punch positions to be corrected it is advantageous when placed markings can be selected and their position altered.

The attachment of comments to the placed markings can later, when the samples are analysed, facilitate the diagnosis.

Specific sample carriers for insertion of the punched-out samples can be assigned to the placed markings, which correspond to individual punch positions. If this feature is not used the available sample carriers can for example be filled sequentially with the punched-out cores.

If specific sample carriers are assigned to the placed markings, specific holes in these sample carriers can furthermore be assigned to the markings. Thus, a layout of the samples most advantageous for subsequent analysis of the micro arrays or tissue arrays can be achieved.

To achieve an unambiguous assignment of the individual tissue samples arranged in the sample carrier during the later analysis of the sections prepared from the tissue samples it is planned to arrange the holes for the tissue samples in the sample carrier in a pattern in which the arrangement of the holes is in the form of a binary code. Consequently, an unambiguous layout of the samples is achieved. In this way the delivery of incorrectly assigned measurements from the section of the sample due to upturning the slide or turning the slide can be prevented. Naturally the samples can be arranged in several different patterns that unambiguously determine the orientation of the micro array.

To achieve that, the samples are punched-out at a predefined depth and it is planned, prior to the punching process, to detect the position of the surface of the preparation and to save the detected position values in association with an identifier for the preparations. The preparations normally have variable heights so their surface must be detected for the achievement of a precise punching depth. Through the storage of identifiers for the preparations together with the detected surface levels an automated manipulation process for a large number of samples can be started.

The detection of the surface can also be carried out for the sample carriers into which the samples are inserted. In this way punched-out holes in the sample carrier of essentially the same depth are always obtained, so that the punched-out samples fit accurately into the holes in the sample carrier. For this the detected surface levels are likewise stored together with identifiers for the sample carriers so that they can be used during the subsequent automated manipulation process.

Advantageously, the punch depth can be selected for the sample punching and hole-punching process and assigned to the markings. In this way different punch depths for different punch positions can be defined or different punch depths selected for different preparations respectively.

The automated punching process is started after placing the final marking on the final preparation. Thereby the preparations are sequentially positioned under the hole-punching needle and sample removed at the stored punch positions which are thereafter inserted into the pre-assigned holes in the corresponding sample carrier. The punching process can be carried out quickly and automatically for a large number of samples.

After at least several punching cycles the needle can be automatically cleaned to remove contaminations from it. For this purpose the sample punching needle and also the hole-punching needle can be automatically moved to a cleaning station where they can be cleaned with an appropriate cleaning fluid and compressed air.

The second task of the invention is solved through the above named device for manipulating samples, in particular tissue samples, for which a camera is provided to take images of the surface of the preparations, further a mechanism for superimposing the captured images of the preparations with digital microscopic images of sections of these preparations stored in a computer memory, further a display to show the superimposed image and a mechanism to specify defined punch positions that is connected with the needle control system. The selection of the desired punch positions can be supported and accelerated through the camera for the recording of surface images of the preparations and the device for superimposing the surface images with the microscopic section images. For this the device for superimposing the images preferably consists of a computer. After placing the defined punch positions the corresponding controller of the hole-punching needle can be appropriately controlled, resulting in an automated probe manipulation.

Because microscopic images that contain detailed information about specific and localised changes of the preparation, in particular tissue changes require appropriate microscopic magnification, it is usually the case that due to the limited display detail of digital microscopic images a complete image of a preparation cannot be depicted in one image. This requires the display of a preparation in multiple segments which can be respectively superimposed on the image of the paraffin block. Furthermore, a device for the assembly of digital microscopic images from multiple segments can be provided. This device can also consist of the computer described above or can alternatively consist of a separate computer or equivalent.

By superimposing multiple image segments that can be positioned independently of each other it is also possible to eliminate stretching artefacts that occur during the production of histological sections.

Usually at least one needle is provided to punch holes in sample carriers, into which the punched-out samples are inserted.

Advantageously a database is provided to store the markings or rather their coordinates together with an identifier for the preparation and if required patient information or equivalent. Said database can for example be integrated with the computer that already represents the device for superimposing the images, but can also be located externally and connected using appropriate data connections with the computer system or equivalent.

In order to achieve an optimal selection of the punch positions devices can be provided to alter the display scale, to alter the orientation, to move or to alter the colouring of the section images, surface images and/or superimposed images respectively.

To enable optimal preparation of the superimposed image and so enable adequate assessment of the preparation, devices can be provided to modify the degree of translucency of the surface images in relation to the microscopic section images and devices to modify the transparency of the microscopic image. Normally the processes described above are carried out using an appropriate computer.

The procedure for placing the markings to determine the definitive punch positions may be carried out using a computer mouse.

To achieve a high quality and specificity off micro arrays and consequently a high quality of the resultant measurements a device to detect the position of the surface level of the sample carrier as well as that of the preparations can be provided. Through the determination of the exact position of the surface of the sample carrier and preparations, the holes in the sample carrier and as well as the samples are always prepared with exactly the defined punch depth.

Thereby a storage device for the detected position values of the sample carrier or preparations together with the identifier for said sample carriers or preparations is advantageous. This storage device can be a part of the database described above.

Advantageously, at least one hole-punching needle and at least one sample punching needle are mounted together on a common pivoting head which is advantageously manipulated using a pneumatic pivot drive. In this way the axes of the hole-punching needle and of the punching needle intersect at the pivot point of the pivoting head. Consequently, a change between the hole-punching and sample punching needle can be achieved by a simple turning of the pivoting head. Further solely one drive unit for the pivoting head must be provided rather than multiple drive units for each needle.

Further, a drive unit is provided to displace the pivoting head relative to the sample carriers or preparations respectively. This may either be located on the pivoting head or on the support for the sample carriers or preparations, so that the displacement of the pivoting head as well as of the needles with respect to the sample carriers or preparations can be accomplished. This drive unit is also advantageously constructed pneumatically.

In order to solve the tasks of the invention a computer programme product, comprising software code sections is used, that may be directly loaded into the internal memory of a digital computer, whereby the computer can carry out the steps of the method described above, when the program is running on the computer.

Thereby, the computer programme product is advantageously stored on a computer-readable medium.

The present invention is further exemplified by the drawings, which demonstrate the principal and embodiments of the invention.

Therein show:

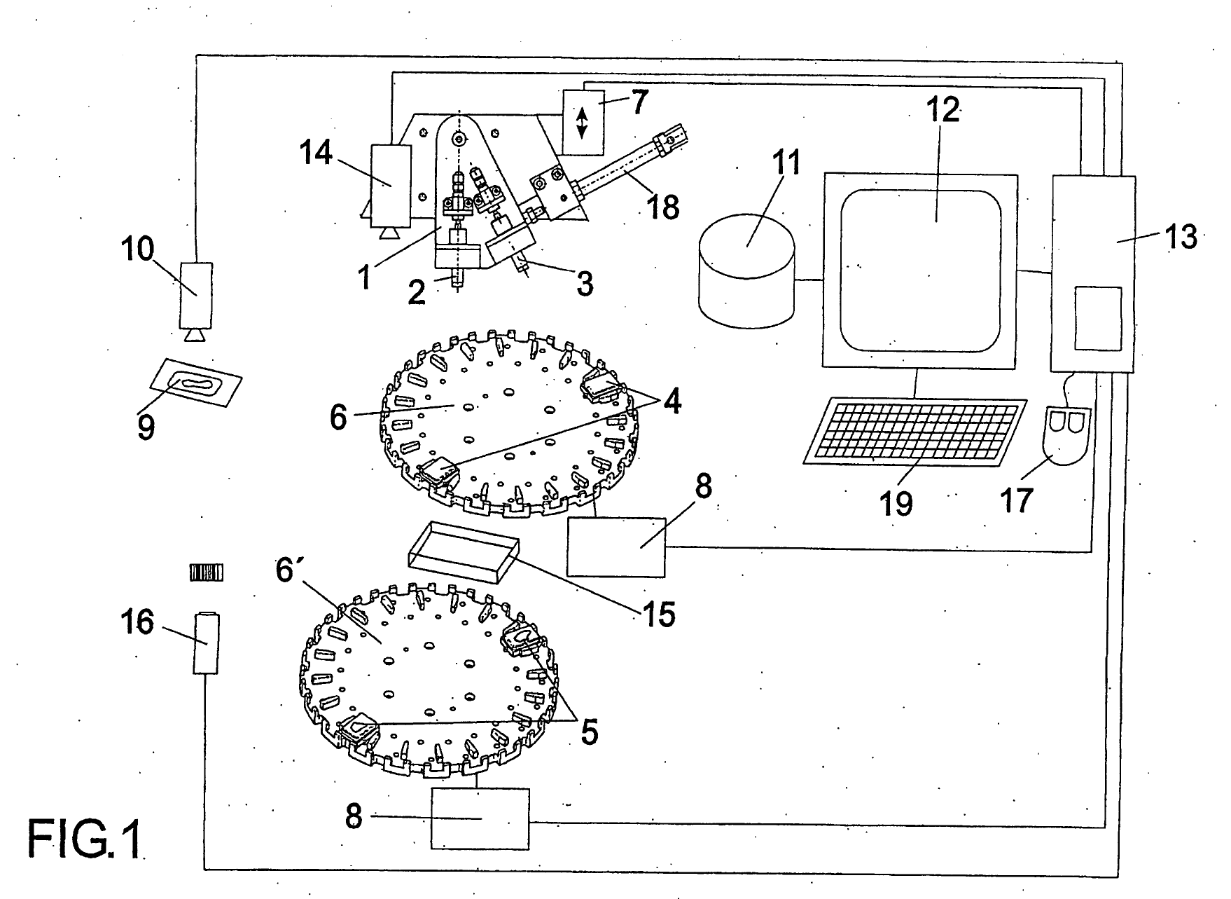

FIG. 1 a schematic illustration of a device for the manipulation of samples, in particular tissue samples;

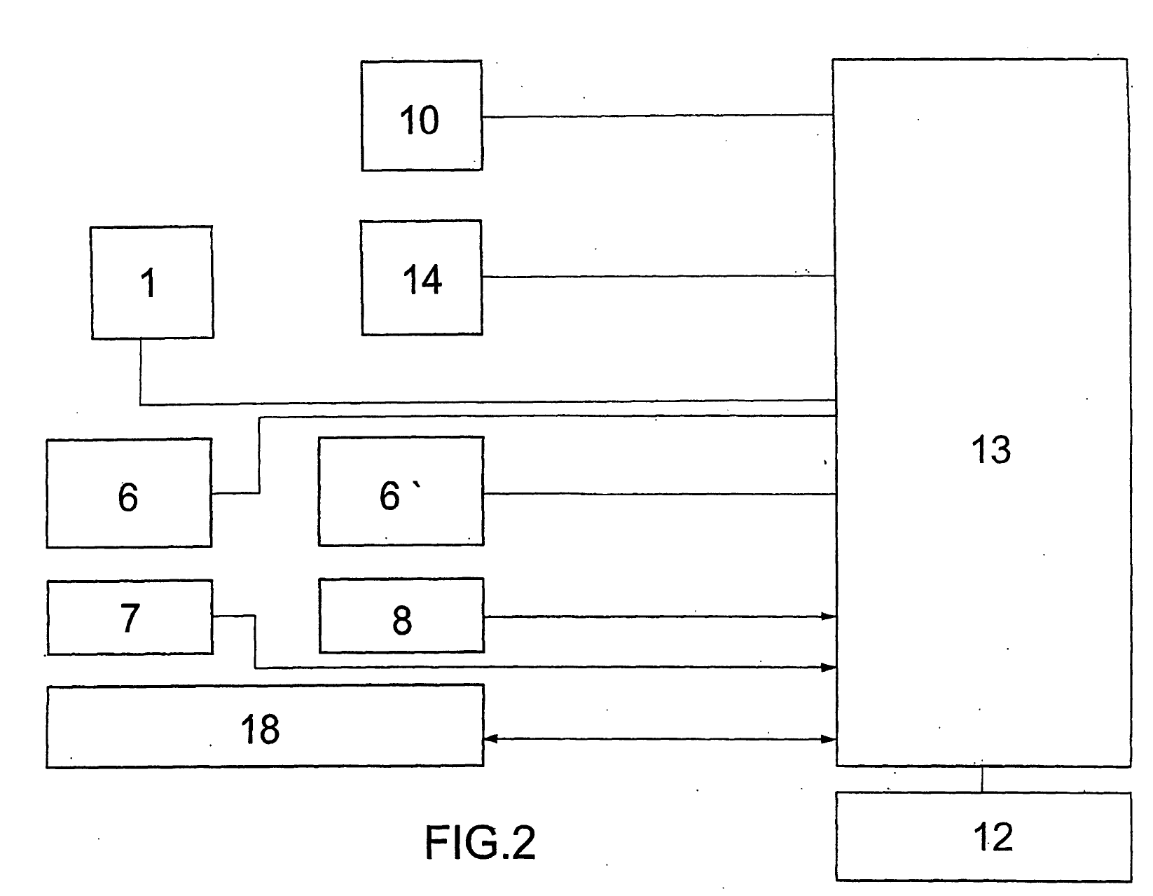

FIG. 2 a schematic block diagram of a device for the manipulation of samples;

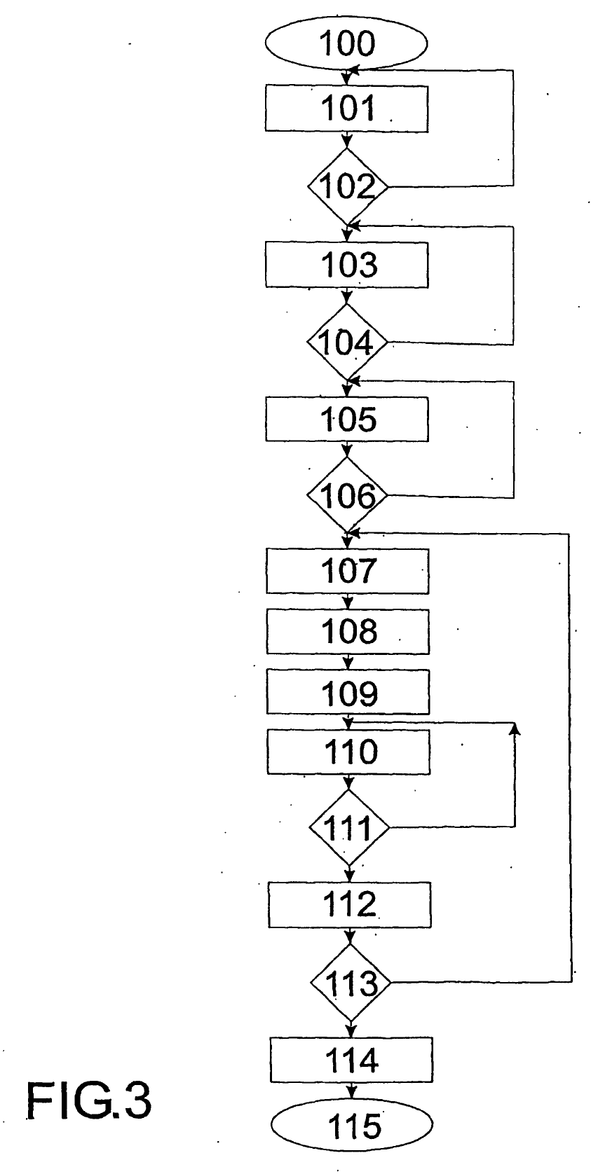

FIG. 3 a flow chart to illustrate the method of the invention for the manipulation of samples; and

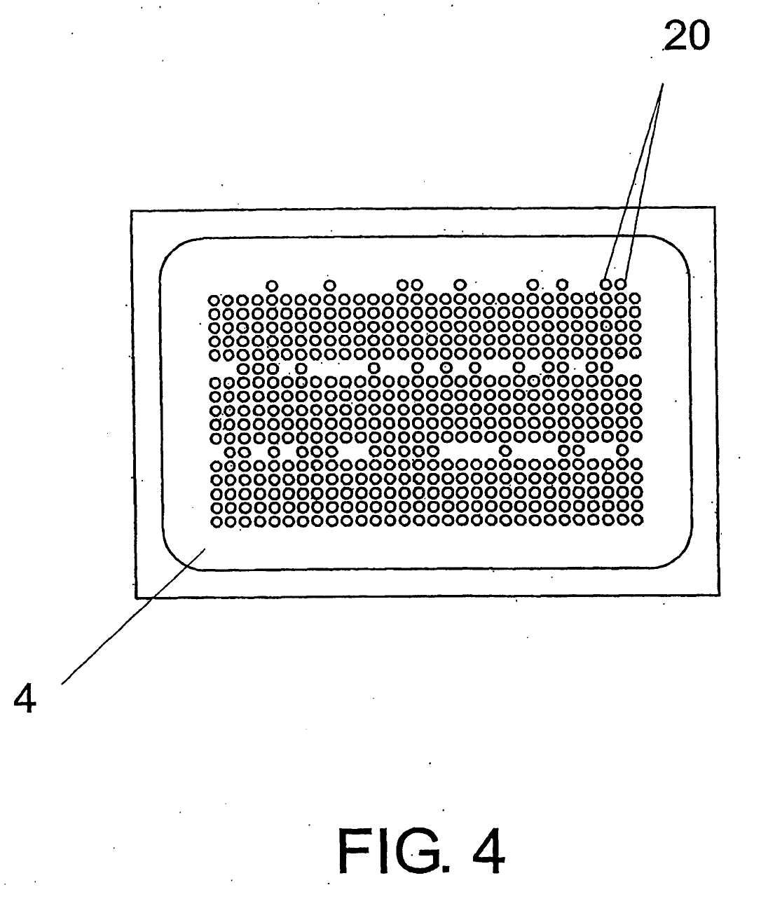

FIG. 4 a top view of an embodiment of a sample carrier containing multiple inserted samples

FIG. 1 shows a schematic illustration of a device for the manipulation of samples, in particular tissue samples. Thereby is a needle 2 for punching holes in the sample carrier 4 and a needle 3 for punching out samples, in particular tissue samples from preparations 5, mounted on a pivoting head 1. Preparations 5 can be biological or animal tissue specimens but can also be other types of samples, for example plants or plant specimens, cell suspensions or bacterial suspensions. The pivoting head is displaceable arranged next to a support 6 on which the sample carriers are placed and a support 6′ on which the preparations are placed so that the needles 2, 3 can be inserted into sample carriers 4 or preparations 5. For this a drive unit 7 for displacing the pivoting head 1 and a drive unit (not shown) for displacing the supports 6, 6′ are provided. The supports 6, 6′ have a circular shape and are arranged next to each other in the illustrated embodiment so that the path of the pivoting head 1 from the respective preparation to the required sample carrier 4 is minimised. Appropriate holders (not shown) are provided for the arrangement of the sample carriers 4 as well as of the preparations 5 on the supports 6, 6′. Selection of the chosen preparation 5 as well as of the chosen sample carrier 4 can be achieved by rotation of the supports 6, 6′ with an appropriate drive unit 8. A container 15 may be provided for receiving ejected material form the sample carriers 4 as well as for cleaning the needles 2, 3 that is located between the supports 6, 6′ for the sample carriers 4 and preparations 5 respectively. The sample carriers 4 and the preparations 5 are usually annotated with a definitive identifier, for example a barcode, which can be read by an appropriate scanner 16. The needles 2, 3 are mounted on the pivoting head 1 so that their axes intersect one another exactly at the pivot point of the pivoting head 1. This ensures that the hole-punching needle 2 and the sample punching needle 3 always come to rest in exactly the same position following a pivot process. Advantageously a pneumatically operated pivoting system 18 is used to turn the pivoting head 1. This pivot system 18, as well as the drive units 7, 8 are linked to a control system, which can for instance be represented by a computer 13. Sections 9 are prepared from each preparation and are photographed using a microscope camera 10. The photographed digital, microscopic images of the sections are then stored in the storage device 11, that is connected with the computer 13. The surface of the preparations 5 is photographed by means of a camera 14 and illustrated on an monitor 12. According to the invention surface images of the preparations 5 that have been captured by means of the camera 14, are superimposed over selected sections images of the same preparations 5 and illustrated on the monitor 12. After the persons skilled in the medical arts have accurately superimposed the appropriate section images and surface images, markings can be placed on the superimposed image, which correspond to the desired punch position of the punching-out needle 3. For that purpose, an appropriate marking can be placed on the monitor 12 for instance with a computer mouse 17. The superimposed images can for instance be altered by means of a computer 13, regarding the display scale, the position, the colouring etc. The placed markings may retrospectively be selected and erased again or moved and with the help of the keyboard 19, additional annotations regarding the markings can be added. The annotations are stored in conjunction with the positions for the markings in a storage device 11. Additionally to the selected punching-out positions for the samples, certain sample carriers 4 can be assigned, into which the samples are inserted. In that way, even specific holes in the sample carriers 4 can be selected for the particular sample. All these data are archived in the storage device 11 or in a different storage device that is connected with the computer 13.

FIG. 2 shows a simplified block diagram of the device for manipulating samples, consisting of a computer 13 that is connected with a monitor 12. Furthermore the computer 13 is connected with the camera of a microscope 10, to take digital, microscopic section images 9 of the preparations as well as a camera 14 to take surface images of the preparations 5. After placing the appropriate markings for the punch positions an automated punching process is started and the pivoting head 1, the turntables 6, 6′ as well as the drive units 7, 8 as well as the pivoting drive 18 of the pivoting head 1 are appropriately controlled by the computer 13. In this manner, a large number of samples can be processed automatically and arranged in appropriate sample carriers 4.

FIG. 3 shows a flow chart illustrating the process of the method according to the invention. After the start according to block 100, the identifier of the first sample carrier 4 is scanned in corresponding to step 101. That is for example carried out by an appropriate scanner that reads the barcode that is displayed on the sample carrier 4. According to scan 102 it is checked if a further sample carrier 4 is provided. The procedure 101 is repeated until all sample carriers have been registered. According to block 103 the height of the first sample carrier 4 is established. As long as further sample carriers are present according to scan 104, step 103 is repeated and the height of the sample carriers 4 established. According to block 105, the size of each sample carrier 4 is determined, by determining the number of samples intended for the sample carrier 4. According to scan 106, block 105 is repeated for all sample carriers 4. According to block 107, the identifier of the first preparation 5 is scanned in. According to block 108, a surface image of the preparation 5 is prepared and shown on a monitor. According to block 109, a particular microscopic image of said preparation 5 is selected from a storage device and superimposed with the surface image captured in block 108. According to block 110, a marking is placed for instance by utilizing a computer mouse, at which a sample is to be punched-out from the preparation 5. According to scan 111, it is examined if further markings should be placed, in which case block 110 will be repeated correspondingly. According to block 112, the sample carrier 4 intended for the samples of the preparation 5 is selected and assigned to the placed markings. The steps according to 107 to 112 are repeated correspondingly for all existent preparations 5. For this purpose, the next preparation 5 is examined according to scan 113. The punching process is started according to block 114, when the last preparation 5 has been processed and all markings placed. The procedure is ended according to block 115, when all samples of all preparations 5 have been arranged in the appropriate sample carriers 4. This flow-chart only shows the main components of the method according to the invention, processing steps for the treatment of the superimposed images to modify the markings and to influence the punching process have not been accounted for.

FIG. 4 shows a top-view of a sample carrier 4 with a total of 487 positions for holes 20 to accommodate 487 samples. In that case the holes 20 are arranged in a pattern that, also after the production of the sections, allows an unambiguous assignment of the samples. In the illustrated example the columns are binary coded with a part of the holes 20. Consequently, it is not possible to confuse the samples by upturning the slide or by turning the slide after the preparation of the sections. Naturally there are numerous other possibilities to achieve such unambiguous assignments.

The present invention offers the information of microscopic images to support the selection of punch positions on the preparations and additionally enables the attribution of the samples to the punch positions on the microscopic image and consequently a better documentation and quality control of the entire procedure.

1.-39. (canceled)

40. A method for manipulating a sample comprising:
superimposing at least one digital, microscopic image of a section of at least one preparation on an image of a surface of the preparation;
placing at least one marking on the superimposed image, which marking defines a desired position at which at least one sample is to be punched out of the preparation;
punching out at least one sample from at least one defined position of at least one preparation with a needle; and
inserting said at least one sample into a hole in a sample carrier comprising at least one hole.

41. The method of claim 40, wherein the sample is a tissue sample.

42. The method of claim 40, wherein the preparation is a prepared tissue specimen.

43. The method of claim 40, wherein the digital, microscopic image comprises several segments assembled prior to superimposing the image on the image of the surface of the preparation.

44. The method of claim 40, wherein the digital, microscopic image has been straightened or had artifacts eliminated prior to superimposing the image on the image of the surface of the preparation.

45. The method of claim 40, wherein the marking and/or its coordinates are saved in a database, together with an identifier for the preparation.

46. The method of claim 40, wherein several digital section images can be selected for each preparation.

47. The method of claim 40, wherein a display scale of section images, surface images and/or superimposed images employed in the method can be modified.

48. The method of claim 40, wherein an orientation of section images, surface images and/or superimposed images employed in the method can be modified.

49. The method of claim 40, wherein section images, surface images, and/or superimposed images employed in the method can be displaced.

50. The method of claim 40, wherein color of section images, surface images, and/or superimposed images employed in the method can be modified.

51. The method of claim 40, wherein a level of translucency of a surface image in relation to a section image can be modified.

52. The method of claim 40, wherein transparency of a section image can be modified.

53. The method of claim 40, wherein patient information or equivalent assigned to an identifier for the preparation, is displayed in combination with the superimposed image.

54. The method of claim 40, wherein the placed marking is characterized.

55. The method of claim 54, wherein the placed marking is numbered consecutively with other placed markings.

56. The method of claim 40, wherein the placed marking can be selected and erased.

57. The method of claim 40, wherein the placed marking can be selected and modified.

58. The method of claim 40, wherein at least one annotation can be assigned to the placed marking.

59. The method of claim 40, wherein a sample carrier for the insertion of the punched-out sample can be assigned to the placed markings.

60. The method of claim 40, wherein a specific hole in the sample carrier can be assigned to the placed marking.

61. The method of claim 40, wherein holes for samples in the sample carrier are arranged in a pattern and said pattern is formed by arrangement of the holes in the form of a binary code.

62. The method of claim 40, wherein a position of the surface of the preparation is detected before the sample punching procedure and detected position values are saved in conjunction with an identifier for the preparation.

63. The method of claim 40, wherein a position of the surface of the sample carrier is detected before the hole-punching procedure and detected position values are saved in conjunction with an identifier for the sample carrier.

64. The method of claim 40, wherein punching depth of the sample punching and hole punching can be selected and assigned to the marking.

65. The method of claim 40, wherein the sample punching is started automatically after the placement of a last marking on a last preparation.

66. The method of claim 40, wherein the sample punching procedure can be interrupted and continued.

67. The method of claim 40, wherein all holes are punched out from the sample carrier before the start of sample punching procedure.

68. The method of claim 40, wherein the sample punching needle and/or hole punching needle are cleaned at least after several sample punchings and/or hole punchings.

69. A device for manipulating samples comprising:
at least one needle for punching samples from preparations at defined positions during use; and
a control device to control the needle during use comprising:
a camera to take images of the surface of the tissue specimen;
a device for superimposing the images captured of the preparations with digital, microscopic images of sections of the preparations that have been archived in a storage device;
a monitor to display the superimposed images; and
a device provided to place markings for the determination of the defined sample-punching positions, which is connected with the control device.

70. The device of claim 69, further comprising a device for assembling the digital, microscopic images of sections of the preparations from several segments.

71. The device of claim 69, further comprising at least one needle to punch holes in sample carriers during use.

72. The device of claim 69, further comprising a database for storing markings and/or their coordinates, an identifier for the preparation, and patient information or an equivalent.

73. The device of claim 69, further comprising a device to alter display scales of section images, surface images, and/or superimposed images.

74. The device of claim 69, further comprising a device to change orientation of section images, surface images, and/or the superimposed images.

75. The device of claim 69, further comprising a device to displace section images, surface images, and/or superimposed images.

76. The device of claim 69, further comprising a device to change color of the section images, surface images, and/or superimposed images.

77. The device of claim 69, further comprising a device to change a level of translucency of surface images in relation to section images.

78. The device of claim 69, further comprising a device to change transparency of the section images.

79. The device of claim 69, wherein the device for placing the markings to determine the defined punch positions is a computer mouse.

80. The device of claim 69, further comprising a device for detecting a position of the surface of the sample carriers and/or preparations during use.

81. The device of claim 80, further comprising a storage device for the detected position values of the sample carriers and/or preparations.
